(19) United States
(12) Patent Application Publication
(10) Pub. No.: US 2025/0174355 A1
(43) Pub. Date: May 29, 2025

(54) METHOD AND SYSTEM FOR MULTI-CANCER MANAGEMENT IN SUBJECT

(71) Applicant: Artificial Intelligence Expert SRL, Cluj-Napoca (RO)

(72) Inventor: Alexandru Floares, Cluj-Napoca (RO)

(21) Appl. No.: 18/868,735

(22) PCT Filed: Jul. 4, 2023
(86) PCT No.: PCT/IB2023/056929

§ 371 Date: Nov. 23, 2024

Related U.S. Application Data
(60) Provisional application No. 63/367,642, filed on Jul. 4, 2022.

(51) Int. Cl. G16H 50/20; G16B 25/10

(52) U.S. Cl. CPC G16H 50/20; G16B 25/10

(57) ABSTRACT

In general terms present invention proposes a method for multi-cancer management in a subject. The method comprises obtaining a miRNA expression profiling raw data; selecting a quality miRNA expression profiling raw data from amongst the obtained miRNA expression profiling raw data; converting the quality miRNA expression profiling raw data into a formatted data; and subjecting the formatted data to an artificial intelligence-based pipeline, for multi-cancer early detection and diagnosis in the subject. The present invention also proposes a system for multi-cancer management in a subject. The system comprises a processor configured to perform steps of the aforementioned method.

### FIELD OF THE INVENTION

This invention relates to artificial intelligence in healthcare. In particular, though not exclusively, this invention relates to a method and a system for multi-cancer management in a subject.

### BACKGROUND

Cancer has been identified as a leading cause of death worldwide, accounting for nearly 10 million deaths per year. Notably, survival rates have improved in the past owing to advancements in cancer research and diagnosis techniques. However, most cancer diagnosis techniques are surgical procedures associated with fear, pain, and risks. They are usually costly, with modest accuracy, and generally applied to patients with cancer symptoms. Moreover, they are harmful to patients and cannot be repeated many times and used for evolution monitoring. Notably, imaging techniques, such as breast mammography, cystoscopy, colonoscopy, used for screening of breast cancer, urethra and bladder cancer, large intestine (colon) and rectum cancer, need a tumor of a specific size to be detected and the related irradiation cannot be neglected, especially for repeated use. Seldom invasive procedures are used for screening. For example, liquid biopsy-based tests are minimally invasive, but they have low or modest accuracy in the early stages. As a consequence of the above listed techniques, cancer is detected mainly in later stages, negatively impacting patients' health and lives, physicians' reputations, healthcare costs, and society.

Conventionally, various standards have been reported for various types of cancer early detection and diagnosis. In an example, the actual standard for breast cancer early detection and diagnosis is the imaging method, namely mammography. Moreover, mammography has its own set of limitations, for example, the use of repeated irradiation, additional testing (such as ultrasound or a tissue biopsy), inaccuracy (i.e. missing 1 in 8 breast cancers), and a high rate of false-negative results (FNR). Moreover, mammography has a sensitivity of 76.5% and a specificity of 87.1% for women <40 years, and the sensitivity and specificity for 75-79 years are 88.4% and 93.5%, respectively. Similarly, the actual standard for lung cancer is usually screening by using the following methods (with the associated performances): X-Ray (77% to 80% sensitivity); Low Dose Computed Tomography (false-positive rate (FPR) of 0.96); and PET scans (74% accuracy, FPR of 39%, and FNR of 9%). Similarly, the actual standard for prostate cancer is usually screening by using the following methods (with the associated performances): Prostate-Specific Antigen (PSA) (4-10 ng/ml PSA range; AUC of 0.53); Prostate-Specific Antigen density (PSAd) (4-10 ng/ml PSA range; AUC 0.70); and Digital Rectal Examination (sensitivity of 51%, specificity of 59%).

Moreover, current tests focus on mutations, methylation, or fragmentation patterns of circulating tumor DNA (ctDNA) or free DNA (cfDNA). However, the low accuracy of these tests indicates that these molecular alterations are not informative enough for the early stages of cancer detection. One such test is developed by GRAIL (San Francisco, California), the Galleri® Multi-Cancer Early Detection test which uses a simple blood draw to identify up to 50 types of cancers. However, test sensitivity of Galleri® for earlier stages is very low, with a meager 16.8% at stage I, rising to just 40.4% at stage II for all cancer types. Furthermore, the Galleri® blood test is expensive and amounts to approximately 1000 USD in price.

Therefore, in the light of the foregoing discussion, there exists a need to overcome the aforementioned drawbacks associated with the conventional ways of multi-cancer detection and provide non-invasive, non-irradiating, non-expensive, highly accurate multi-cancer early detection tests.

### SUMMARY OF THE INVENTION

A first aspect of the invention provides a method for multi-cancer management in a subject, the method comprising:
(a) obtaining a miRNA expression profiling raw data;
(b) selecting a quality miRNA expression profiling raw data from amongst the obtained miRNA expression profiling raw data;
(c) converting the quality miRNA expression profiling raw data into a formatted data; and
(d) subjecting the formatted data to an artificial intelligence-based pipeline, for multi-cancer early detection and diagnosis in the subject.

Suitably, the method of the present disclosure provides a non-invasive AI-powered multi-Cancer early detection test, based on circulating miRNA. In this regard, the method employs miRNA expression profiling raw data obtained from various miRNA profiling technologies (such as Microarray, Next Generation Sequencing (NGS), Polymerase Chain Reaction (PCR), or Nanostring) and submits them to AI-based predictive models for multi-cancer early detection and diagnosis in the subject. Moreover, the method outputs the diagnosis (cancer or normal), the cancer localization (or type), and the corresponding confidence. Furthermore, the method provides a personalized diagnosis that is understandable by physicians including the individual molecular alterations (such as signaling and metabolic pathwyas, or protein-protein-interaction networks), potential treatments, and so on, supporting the suggested diagnosis. Additionally, the method holds up to a >99%, and for some cancer types, even 100%, accuracy across all stages compared to the conventional cancer detection and diagnosis techniques.

Throughout the present disclosure, the term “multi-cancer management” as used herein refers to liquid biopsies for early detection and diagnosis of multiple cancer types in the subject via multiple biomarkers (such as circulating tumor cells, tumor DNA/RNA and so on, in blood or other body fluids) of a growing cancer. Moreover, the multi-cancer management may also extend to monitoring the subject's response to the treatment prescribed based on the detection or diagnosis of multiple cancer in the subject's body to provide precision and personalized medicines for the subject. Herein, the term “subject” refers to a person (human being) with or without cancer symptoms of a growing cancer therein. Optionally, the subject may have been diagnosed with cancer. Optionally, the subject may not be showing cancer symptoms and is required to undergo a screening for confirming the same.

Herein, the term “miRNA” refers to a class of small single-stranded non-coding RNA molecules that play an important role in the post-transcriptional regulation of gene expression. miRNAs act on their target mRNAs mainly to induce mRNA degradation and translational repression. In this regard, the miRNAs base pair with complementary sequences within mRNA molecules and result in the silencing of such mRNA molecules by one or more processes. Typically, miRNA (abbreviation for microRNA) comprises an average of 22 nucleotides in length.

In an embodiment, the miRNA is circulating miRNA. The miRNAs, besides other origins, are also actively secreted into the extracellular fluids. The extracellular miRNA (namely, the circulating miRNAs) serve as potential biomarkers for a variety of diseases. Optionally, the extracellular fluids may include, but do not limit to, plasma and serum, cerebrospinal fluid, saliva, breast milk, urine, tears, colostrum, peritoneal fluid, bronchial lavage, seminal fluid, and ovarian follicular fluid. In the extracellular fluids, the miRNA may be associated with exosomes, microvesicles, apoptotic bodies, and proteins. Beneficially, circulating miRNAs are highly stable and resist degradation at room temperature for up to 4 days, and can withstand deleterious conditions, such as high temperatures, free-thaw cycles, and high or low pH.

Notably, miRNAs serve as signaling molecules to mediate cell-cell communications and as modulators of cellular activities. In this regard, the miRNAs may bind to Toll-like receptors, activate downstream signaling events, and eventually lead to biological responses, such as tumor growth and metastasis, neurodegeneration, and so on. Moreover, miRNAs do not require a high complementarity for regulation, therefore, a single miRNA may target up to several hundred mRNAs and the resulting aberrant miRNA expression may affect a multitude of transcripts, which have a profound influence on cancer-related signaling and/or metabolic pathways (comprising various oncogenes or tumor suppressor genes). Such cancer-related signaling and/or metabolic pathways may optionally include, but do not limit to pathways involved in causing breast cancer, colon cancer, gastric cancer, lung cancer, prostate cancer, and thyroid cancer. Therefore, knowledge of such miRNAs is very important in understanding their role in cancer diagnosis and therapy.

Herein, the term “miRNA expression profiling raw data” refers to a dataset comprising measurement of the expression levels of miRNAs. The expression profiling of miRNAs has already entered cancer clinics as diagnostic and prognostic biomarkers to assess tumor initiation, progression, and response to treatment in cancer patients. Notably, miRNA expression profiles are more accurate in disease classification than mRNA expression profiles.

In an embodiment, the miRNA expression profiling raw data is obtained from any of: Next Generation Sequencing technology, Polymerase Chain Reaction technology, Nanostring technology, or Microarray technology. It will be appreciated that the aforementioned miRNA expression profiling technologies serve as high-throughput tools for a better understanding of the biological functions of miRNAs and using miRNA expression as potential diagnostics. Such technologies have enabled accurate quantification of miRNAs and the understanding of their role in various biological processes. Beneficially, the Nanostring technology is operable with a wide range of biological samples, including some poor-quality biological samples, to address novel treatment options in cancer, for example.

Herein, the Polymerase Chain Reaction technology (PCR), such as reverse transcription quantitative PCR (RT-qPCR), is the most commonly used, cost-effective, sensitive, and reproducible method for relative gene expression quantification. Notably, high-throughput real-time quantitative reverse transcriptase polymerase chain reaction (qPCR) is widely accepted to be “a gold standard” in regard to gene expression analysis. Moreover, a normalization of RT-qPCR data is required to ensure reliable results as compared to the gold-standard RT-qPCR technology. Herein, the Microarray technology is capable of monitoring the expression of thousands of miRNAs from multiple samples in a single experiment. In this regard, the Microarray technology incorporates different design strategies to improve the specificity of probes, such as nucleic acid-based probes, for miRNAs during labeling in miRNA arrays. Optionally, results from one technology may be validated using another technology, for example, results of the Microarray technology may be validated using PCR (RT-qPCR) technology. However, the Microarray technology may suffer from background signal and cross-hybridization issues.

Herein, the Nanostring technology refers to an amplification-free technology for miRNA expression profiling by direct quantitation of individual miRNA molecules. It detects miRNAs without use of reverse transcription or amplification by using molecular barcodes, thereby making it easier and faster to validate miRNA biomarkers as compared to other quantitation techniques. The Nanostring technology gives reproducible results with high specificity from low amounts of biological samples. However, the Nanostring technology is associated with low sensitivity. Herein, the Next Generation Sequencing technology provides the highest sensitivity, accuracy and the largest dynamic range of detection of miRNAs expression. Moreover, the Next Generation Sequencing technology avoids any cross-hybridization issues, thus offers a more comprehensive view of the miRNA expression profiling.

It will be appreciated that other publicly available miRNA expression profiling data repositories using different platforms may also be used. Moreover, miRNA expression profiling counts vary based on the type of biological sample and the miRNA expression profiling technology used. Optionally, the miRNA expression profiling raw data may be normalized and scaled from high-throughput studies by ways known to a person skilled in the art.

In an embodiment, the method further comprises performing at least one of: a quality control step, a filtering step, an adaptor trimming step, an alignment step, a quantification step, a functional analysis step. In an exemplary implementation, analyzing miRNA expression profile using the Next Generation Sequencing technology requires a technology specific standardized bioinformatics pipeline. In this regard the Next Generation Sequencing technology data requires several transformation steps before they can be used as an input in the artificial intelligence-based pipeline. Such steps include the quality control check, the filtering, the adaptor trimming, the alignment step, the quantification and, optionally, differential miRNA expression (e.g., between Cancer and Normal) and the functional analysis steps.

It will be appreciated that not all miRNA expression profiling raw data is required to undergo further analysis, and therefore quality miRNA expression profiling raw data is selected from amongst the obtained miRNA expression profiling raw data. Herein, the term “quality miRNA expression profiling raw data” refers to a dataset comprising measurement of the expression levels of miRNAs that are associated with one or more diseases (altered miRNA) that are different from a normal cohort. In this regard, the quality miRNA expression profiling raw data may be associated with a defined miRNA integrity, quantity, and contamination-free miRNA. Herein, the quality of the sequencing files (miRNA expression profiling raw data) is checked and a detailed report with all information related to the sequences is produced. The content for each individual case is then merged in a general report including all probes from the experiment detailing upon various sequence quality metrics (nucleotides, GC content, error rate, presence of adapter sequences). The report is then analyzed, and decisions are taken regarding any further filtering steps.

It will be appreciated that the trimming step allows to set a maximum read length, i.e., an average length of 22 nucleotides (O'Brien et al. 2018), that is very useful in filtering the molecules that are not miRNA. In this regard, during the adaptor trimming step, a cutoff for sequence length can be introduced, since miRNA have an average length of 22 nucleotides, a cutoff between 22-30 nucleotides will be set to keep only the molecules of interest and to reduce computing times in the alignment step. Moreover, subjecting the post-quality control data to go through an alignment process against a reference genome (e.g., RCH38—Database: Human reference genome sequence) and against a mature miRNA from miRNA databases (e.g., miRBase v22) and the miRNA quantification and counts generation post alignment step generate files that may be used as an input in the artificial intelligence-based pipeline. Optionally, an additional statistical analysis of the data may be performed for generating a detailed report of the miRNA molecules that is human-readable.

In another exemplary implementation, analyzing miRNA expression profile using the Microarray technology requires the miRNA expression profiling raw data produced by the array scanner software. These miRNA expression profiling raw data contain the measured probe intensities. Subsequently, the miRNA expression profiling raw data is pre-processed, and normalized.

In this regard, high-throughput miRNA expression profiling raw data is obtained from at least one high-throughput miRNA expression profiling technique as mentioned above. Typically, a test is prescribed by a physician or requested by a subject. The biological sample is collected from the subject and sent to a laboratory capable of miRNA expression profiling using any of the aforementioned quantitation techniques. Subsequently, the total RNA is extracted from the biological samples from subjects and miRNA expression within the biological sample is detected and provided as miRNA expression profiling raw data.

Moreover, the method comprises converting the quality miRNA expression profiling raw data into a formatted data. The present invention initiates upon receiving the miRNA expression profiling raw data from the laboratory test and choosing only the quality miRNA expression profiling raw data therefrom. In an embodiment, the method further comprises subjecting the quality miRNA expression profiling raw data to a standardized bioinformatics pipeline specific for a miRNA expression profiling technology. The quality miRNA expression profiling raw data is subsequently provided as an input to specific standardized bioinformatic pipeline (namely, in-silico biological and functional analysis techniques) for further analysis of the quality miRNA expression profiling raw data, to determine the role of a given miRNA on one or more genes thereby resulting in one or more disease phenotypes. The specific standardized bioinformatic pipeline is further configured to convert the input data into a suitable formatted data that may be further used by the artificial intelligence-based pipeline, for the Multi-Cancer AI test, as discussed below. Optionally, a data normalization may be performed before feeding the said data into the AI-based pipeline.

Herein, the term “formatted data” refers to a structure of data according to pre-defined specifications for computer processing. Beneficially, the formatted data enhances presentability of information as well as maximizes reuse of the information. It will be appreciated that the formatted data should be acceptable by the next-in-line machine for further processing of information. Typically, the formatted data is stored in a computer-readable file. In an embodiment, the formatted data is implemented as a CSV (comma-separated values) file, which is a plain text file that contains a large amount of data typically separated by a comma. The CSV file is suitable for exchanging data between different applications. In this regard, typically, complex data from one application (standardized bioinformatic pipeline) is exported to a CSV file, and then the data in the CSV file is imported into another application (artificial intelligence-based pipeline). Beneficially, the resulting data in CSV files is human-readable and can be easily viewed with, for example, Notepad or Microsoft Excel. Optionally, the CSV file may have a specific format which allows data to be stored in a table-structured format.

In an embodiment, the formatted data comprises a table having a plurality of rows and a plurality of columns, wherein each row represents a case, and each column represents a variable. It will be appreciated that a tabular representation of the formatted data assorts the data or information in the plurality of rows and columns of a table. Herein, the term “variable” refers to the recorded data in the formatted data. Typically, the plurality of rows comprise data corresponding to a case or a data record. In an exemplary CSV file, the data corresponding to each case is provided in a specific line separated by a comma, wherein every line (except the last line) in the CSV file ends in a delimiter (such as line breaks (CRLF)). Optionally, the first line of the CSV file contains field names associated with the variables. It will be appreciated that the number of field names is equal to the number of variables.

In an embodiment, the variable is
a case identification
an input represented by miRNA expression profiling raw data; and
an output representing a normal or a cancer of certain type pathologically confirmed diagnostic.

In this regard, the variables are the inputs and outputs for the artificial intelligence-based pipeline to infer the best relationship between the inputs and the outputs. In an example, a first column comprises a case identification (caseID), i.e., information corresponding to the subject (patient or control) for tracking a case, herein the caseID is neither an input nor output. It will be appreciated that such caseIDs serve as metadata, i.e., some useful patient's data, but may not directly be used for developing the predictive model, like the inputs associated with the caseID. Optionally, the inputs may be the one or more miRNAs identified by the various miRNA expression profiling techniques from the biological samples of the patients. Notably, the inputs, namely the miRNAs, are used for early detection, diagnosis and treatment response monitoring. It will be appreciated that the inputs, namely the miRNAs, may also be used for predicting a cancer condition, and predicting a response to treatment. In this regard, a change in the output or having multiple outputs enable the disclosed method to be applied to a larger category of problems. For example, the outputs may include both the diagnosis and the response to treatment. Optionally, the output could be a topic of prediction, such as predicting (or monitoring) a cancer condition (Normal, Breast Cancer, Colorectal Cancer, and so on), and a response to treatment. Optionally, the output is a diagnosis of the patient's current condition.

The method comprises subjecting the formatted data to the artificial intelligence-based pipeline, for multi-cancer early detection and diagnosis in the subject. Herein, the term “artificial intelligence-based pipeline” refers to a set of self-contained codes that perform one-step at a time in a pipeline's workflow, such as data gathering, data processing, data transformation, data flow to downstream systems (or applications), model training, output prediction, and so on, based on a set of inputs. Optionally, Machine learning (ML) based pipeline or a combination of Machine learning and artificial intelligence (ML/AI) based pipelines may be used for the said purpose. Herein, the AI-based, ML-based and the ML/AI-based pipelines include steps to prepare and analyze data, train and evaluate models, deploy such models to production, and so on, to help automate, manage and improve monitoring multi-cancer early detection and diagnosis in the subject. Beneficially, with such data pipelines, the desired multi-cancer early detection and diagnosis in the subject can be drawn within a fraction of seconds and reproduced inexpensively. Additionally, beneficially, application of such data pipelines may be extended further to other analytical processes including, but not limited to, drug discovery and predicting a course of treatment, as would be evident to a person skilled in the art.

In an embodiment, the artificial intelligence-based pipeline is a trained artificial intelligence-based model or an explainable artificial intelligence-based model. Typically, an artificial intelligence-based pipeline may employ 1) an AI algorithm that could learn from data (retraining), or 2) a trained (no learning or “frozen”) AI model applied to the patient's data. Optionally, the AI-based pipeline could be just a model or a combination of models (model amalgamation). The explainable AI (XAI) based model is used to explain the prediction of the above models or amalgamations. Notably, the XAI-based model can work with any kind of AI/ML models or amalgamations to make the intelligence-based pipeline intelligible, transparent, and so on. Moreover, the XAI-based model enables identifying the personalized relevant miRNAs and a mechanistic explanation at the molecular level, based on the results of the functional analysis step.

In an embodiment, the method further comprises repeating steps (a) to (d) at a pre-defined time interval for identifying an altered miRNA of the subject for monitoring a response to treatment. Herein, the term “altered miRNA” as used herein refers to a miRNA (namely, a differentially expressed miRNA) in a patient that is different from a corresponding miRNA in a normal cohort. Typically, the altered miRNA results from an environmental or genetic stimuli, often early during the disease. In this regard, repeating the aforementioned method at predefined time intervals during the course of therapy enables the investigator, such as a doctor or researcher, to monitor a response to treatment. It will be appreciated that the method may be repeated as many number of times during the course of treatment as well as post treatment. Optionally, the term “pre-defined time interval” refers to a period such as every 15 days, or 1 month or 6 months at which the above method could be repeated to track the response to treatment and/or progression of the cancer.

It will be appreciated that there are a couple of possible, well-defined, evolutions of cancer under treatment. Progression and recurrence are just examples of bad evolution, which should be detected early and precisely to help doctors to adjust the treatment to the individualized or personalized patient's evolution. The disclosed method enables detection of all these kinds of evolutions under treatment.

In an exemplary implementation, the patient receives a treatment for example a surgery. After a while, the test may be repeated to estimate the efficacy of the treatment, i.e., by measuring again the circulating miRNAs levels. Optionally, during further testing, new miRNAs values may be introduced in a graphical user interface of the test (namely, the XAI-based model). Notably, the personalized model will be automatically applied to the new data resulting in a new diagnosis and its confidence (or probability). The test's result, if the treatment is effective, could be ‘Normal’, with a high probability. If the treatment was not very effective, the test's result can be ‘Cancer (as initially)’ but with a lower probability, or ‘Normal’ with low probability), indicating a Minimal or Molecular Residual Disease. After a while, a disease recurrence may be suspected, and thus the test is repeated. In such cases, the test's result can be any of: ‘Initial Cancer’, ‘Good initial response to treatment’, ‘Normal’, and ‘Cancer recurrence’. Therefore, after repeating the test and looking at the diagnosis and its probability, the response to treatment can be evaluated even 6-7 months earlier than the conventional methods (such as Computed Tomography and RECIST 1.1 criteria (based on increase/decrease of the tumour size). As mentioned above, the test could be repeated as many times as needed, as cancer management is complex and takes a long time, and the test is based on liquid-biopsy data (non-invasive). At any repeat, a new diagnosis and its confidence (or probability) is obtained and compared with the previous ones.

In an embodiment, the method further comprises mapping the altered miRNA of the subject onto signalling and/or metabolic pathways regulated thereby (functional analysis). It will be appreciated that not all miRNAs are mapped and only the differentially expressed ones obtained after the standardized bioinformatics pipeline. However, alternatively, the AI-based pipeline (such as the XAI-based model) may use the miRNAs with predictive values. Therefore, by using the XAI-based model, relevant miRNAs at the population, group, and individual levels may be identified. Beneficially, such mapping of the altered miRNA onto signalling and/or metabolic pathways regulated thereby enables identification of the degree of impact of such altered miRNA on the signalling and/or metabolic pathways. It will be appreciated that mapping the altered miRNAs onto the signalling and/or metabolic pathways they regulate, and analysing them for druggability and/or network pharmacology thereof towards various molecules (such as proteins targeted by the drug) on the signalling and/or metabolic pathways, may generate reports that may be shared with physicians or doctors or researchers for further interpretation.

In an embodiment, monitoring the response to treatment results in a normal result or cancer recurrence, with a high probability or a low probability thereof. It will be appreciated that the resulting normal result or cancer recurrence, with a high probability or a low probability thereof, are the outputs or outcomes of the artificial intelligence-based pipeline based on the various inputs in the formatted data. Optionally, the method is configured to generate results that may indicate more evolution of cancer under treatment, like progressive cancer, as assessed by RECIST 1.1 Criteria.

The application of the disclosed method extends to cancer localization, i.e., if cancer is the diagnosis, the test will also localize cancer with >99%, preferably 99-100%, accuracy. Notably, the disclosed method provides cancer localization with 99-100% accuracy for 32 cancer types, of which:
(i) 13 are most frequent cancer types, such as Breast Cancer (Breast), Metastatic Breast Cancer (Breast, Metastasis), Ovarian Cancer (Ovaries), Pancreatic Cancer (Pancreas), Colorectal Cancer (Colon, Rectum), Lung Cancer (Lungs), Gastric Cancer (Stomach), Bladder Cancer (Bladder), Esophageal Cancer (Esophagus), Biliary Tract Cancer (Bile Ducts, Gallbladder), Hepatocellular Carcinoma (Liver), Sarcoma (Soft Tissues, Bones) and Glioma (Brain); and
(ii) 19 are relatively less frequent cancer types, such as Prostate Cancer (Prostate), Esophageal Squamous Cell Carcinoma (Esophagus), Oral Squamous Cell Carcinoma (Mouth), Nasopharyngeal Carcinoma (Nasopharynx), Head and Neck Tumor (Head, Neck), Malignant Bone and Soft Tissue Tumor (Bones, Soft Tissues), Multiple Myeloma (Bone Marrow), Lung Adenocarcinoma (Lungs), Lung Squamous Cell Carcinoma (Lungs), Small Cell Lung Carcinoma (Lungs), Stomach Cancer (Stomach), Esophagus Cancer (Esophagus), Liver Cancer (Liver), Intrahepatic Cholangiocarcinoma (Bile Ducts), Gastrointestinal Stromal Tumors (GI Tract), Glioblastoma (Brain), Primary Central Nervous System Lymphoma (Brain, Lymphoma), Brain Metastasis (Brain, Metastasis), and Retinoblastoma (Retina, Eye.

For a given cancer type diagnosis (e.g., Breast Cancer), the disclosed method also gives a confidence score of identifying a particular cancer type (e.g., Breast Cancer with 90% confidence).

Optionally, the application of the disclosed method may further be extended to identifying a personalized altered miRNAs subset relevant for the subject, and repeating the test for measuring just this subset instead of the whole miRNA panel. Beneficially, such a limited subset for measurement is cheaper and faster.

In an embodiment, the method further comprises storing the miRNA expression profiling raw data in a database. Typically, the database is a storage media that may store information either temporarily or permanently. Generally, the database stores data, files, and the like in any format. Herein, the database may be a cloud-based database or an external device that stores the miRNA expression profiling raw data and other data for the purpose of future reference or to run a detailed analysis on the stored miRNA expression profiling raw data and other data for generating further inferences. Thus, the method may be used to compare the test analysis between two or more separate cycles. It will be appreciated that the database is configured to store the miRNA expression profiling raw data and other data that may be used by the ML/AI algorithms for training the dataset.

In an embodiment, the method further comprises predicting a treatment response, based on the multi-cancer early detection and diagnosis in the subject. In this regard, the AI/XAI-based method that can be used to discover and/or design new drugs, that are more likely to be effective and less likely to have side effects, by screening large libraries of compounds for potential drug targets, may also be used to predict the treatment response corresponding to the new drugs for use as personalized cancer treatment. Moreover, the AI/XAI-based method may be used to monitor the effectiveness of treatment and to adjust treatment plans as needed. In this regard, optionally, the AI/XAI-based method comprises collecting a clinical dataset (having information on the patient's cancer type, stage, treatment history, and response to treatment, other medical conditions, co-morbidities, etc.) and identifying features (patterns and trends) in the clinical dataset that could be used to predict treatment response. The AI/XAI-based method (or pipeline) is trained on the identified features from the clinical data, and deployed to help doctors predict treatment response in their patients.

Optionally, the AI/XAI-based method may be tested on a separate dataset of clinical data to assess its accuracy in predicting treatment response.

A technical benefit of predicting the treating response is that it can be used to personalize a treatment for a patient or subjects who are at high risk of developing cancer, so that they can be monitored more closely and receive preventive treatment if necessary. Notably, a suitable treatment regime that aims at selecting and targeting a drug to specific signalling and/or molecular pathways that are driving cancer growth, can improve the chances of a successful outcome.

In an embodiment, the method further comprises providing one or more personalized treatment recommendations for the subject to choose from. It will be appreciated that the personalized treatment recommendations are based on individual characteristics, such as the patient's medical history, test results, genetic profile, lifestyle factors, and preferences. The process of providing personalized treatment recommendations typically involves assessing the subject's medical condition based on factors such as diagnosis, disease stage, and any specific biomarkers or genetic mutations; collecting relevant information including medical records, diagnostic tests, imaging results, and genetic profiling about the subject; analysis of the relevant information by the medical professionals or specialized algorithms to identify patterns, correlations, and potential treatment options based on the subject's specific characteristics; based on the analysis, generating a range of treatment options based on factors like the effectiveness of different therapies, potential side effects, and the subject's individual circumstances; communicating the potential personalized treatment recommendations to the subject to selected therefrom; and receiving a response from the subject or the medical health professional regarding selection of one or more of the communicated potential personalized treatment that aligns with their preferences, values, and goals. Optionally, the step of assessing the subject's medical condition, is based on a training of the AI/XAI-based pipeline/method on a dataset of clinical data that includes information on the patient's cancer type, stage, treatment history, and response to treatment. Optionally, the potential personalized treatment is communicated along with detailed explanations and discussions about the pros and cons thereof.

A technical benefit of the providing one or more personalized treatment recommendations is that they are tailored to the individual and are meant to optimize treatment outcomes by considering the unique characteristics of each subject, thereby leading to more effective and patient-centred care.

A second aspect of the invention provides a system for multi-cancer management in a subject, the system comprising a processor configured to:
(a) obtain a miRNA expression profiling raw data;
(b) select a quality miRNA expression profiling raw data from amongst the obtained miRNA expression profiling raw data;
(c) convert the quality miRNA expression profiling raw data into a formatted data; and
(d) subject the formatted data to an artificial intelligence-based pipeline, for multi-cancer early detection and diagnosis in the subject.

Various embodiments and variants disclosed above apply mutatis mutandis to the system.

Suitably, the system of the present disclosure is a possible replacement, being non-invasive and more accurate, but can also work in combination with other conventional cancer detection and diagnostic techniques such as mammography, and so on.

Throughout the present disclosure, the term “processor” as used herein refers to hardware, software, firmware or a combination of these. Examples of the processor include, but are not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the processor may refer to one or more individual processors, processing devices and various elements associated with a processing device that may be shared by other processing devices. Additionally, one or more individual processors, processing devices and elements are arranged in various architectures for responding to and processing the instructions that drive the system. Herein the processor is operable to obtain a miRNA expression profiling raw data, select a quality miRNA expression profiling raw data from amongst the obtained miRNA expression profiling raw data, convert the quality miRNA expression profiling raw data into a formatted data, and subject the formatted data to an artificial intelligence-based pipeline, for multi-cancer early detection and diagnosis in the subject, as discussed above.

In particular, the processor is coupled to and controls operation of various components of the system and other devices communicably coupled to the aforementioned system. Optionally, the processor also encompasses software and the memory including the computer-executable program code that makes the act of serving information or providing services possible. It may be evident that the communication means of an external device may be compatible with a communication means of the processor, in order to facilitate communication therebetween.

In an embodiment, the processor is further configured to identify an altered miRNA of the subject for monitoring a response to treatment.

In an embodiment, the processor is configured to result in a normal result or cancer recurrence, with a high probability or a low probability thereof, for monitoring the response to treatment.

In an embodiment, the artificial intelligence-based pipeline is a trained artificial intelligence-based model or an explainable artificial intelligence-based model.

In an embodiment, the miRNA expression profiling raw data is obtained from any of: Next Generation Sequencing technology, Polymerase Chain Reaction technology, Nanostring technology, Microarray technology.

In an embodiment, the system further comprises a database for storing the miRNA expression profiling raw data.

In an embodiment, the processor is further configured to subject the quality miRNA expression profiling raw data to a standardized bioinformatics pipeline specific for a miRNA expression profiling technology.

In an embodiment, the processor is configured to perform at least one of: a quality control step, a filtering step, an adaptor trimming step, an alignment step, a quantification step, a functional analysis step.

In an embodiment, the formatted data is implemented as a CSV file.

In an embodiment, the formatted data comprises a table having a plurality of rows and a plurality of columns, wherein each row represents a case and each column represents a variable.

In an embodiment, the variable is
a case identification
an input represented by miRNA expression profiling raw data; and
an output representing a normal or a cancer of certain type pathologically confirmed diagnostic.

In an embodiment, the system is further configured to predict a treatment response, based on the multi-cancer early detection and diagnosis in the subject.

In an embodiment, the system is further configured to provide one or more personalized treatment recommendations for the subject to choose from.

A third aspect of the invention provides a computer program product comprising a non-transitory computer-readable storage medium having computer-readable instructions stored thereon, the computer-readable instructions being executable by a processor to execute a method as claimed in the aforementioned first aspect.

Various embodiments and variants disclosed above apply mutatis mutandis to the computer program product.

Optionally, the computer program product is implemented as an algorithm, embedded in a software stored in the non-transitory computer-readable storage medium. The non-transitory computer-readable storage medium may include, but is not limited to, an electronic storage device, a magnetic storage device, an optical storage device, an electromagnetic storage device, a semiconductor storage device, or any suitable combination of the foregoing. Examples of implementation of computer-readable storage medium, but are not limited to, Electrically Erasable Programmable Read-Only Memory (EEPROM), Random Access Memory (RAM), Read Only Memory (ROM), Hard Disk Drive (HDD), Flash memory, a Secure Digital (SD) card, Solid-State Drive (SSD), a computer readable storage medium, and/or CPU cache memory.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is a flowchart of steps of a method for multi-cancer management in a subject, in accordance with an embodiment of the invention;

FIG. 2 is a schematic illustration of steps of a method for multi-cancer management in a subject, in accordance with another embodiment of the invention;

FIGS. 3, 4, 5, and 6 are flowcharts of miRNA expression profiling analysis using the Next Generation Sequencing technology, Microarray technology, the Polymerase Chain Reaction technology, and the Nanostring technology, respectively, in accordance with various embodiments of the invention;

FIG. 7 is a flowchart of a method of applying an AI-based pipeline workflow for developing and training new Models, in accordance with an embodiment of the invention; and

FIG. 8 is a flowchart of a method of applying a trained AI-based pipeline workflow to the input data from the standardized bioinformatics pipeline, in accordance with an embodiment of the invention.

### DETAILED DESCRIPTION

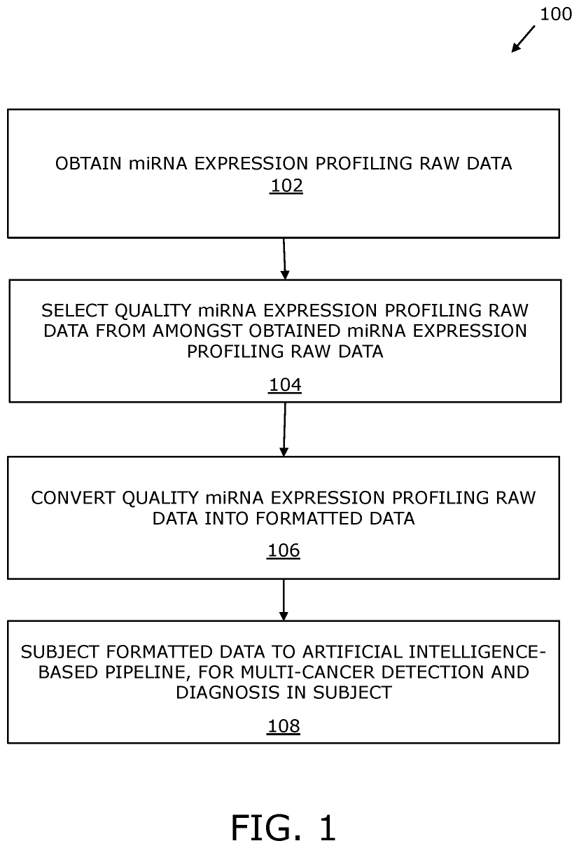

Referring to FIG. 1, shown is a flowchart 100 of steps of a method for multi-cancer management in a subject, in accordance with an embodiment of the invention. At step 102, a miRNA expression profiling raw data is obtained. At step 104, a quality miRNA expression profiling raw data is selected from amongst the obtained miRNA expression profiling raw data. At step 106, the quality miRNA expression profiling raw data is converted into a formatted data. At step 108, the formatted data is subjected to an artificial intelligence-based pipeline, for multi-cancer early detection and diagnosis in the subject.

The steps 102, 104, 106 and 108 are only illustrative and other alternatives can also be provided where one or more steps are added, one or more steps are removed, or one or more steps are provided in a different sequence without departing from the scope of the claims herein.

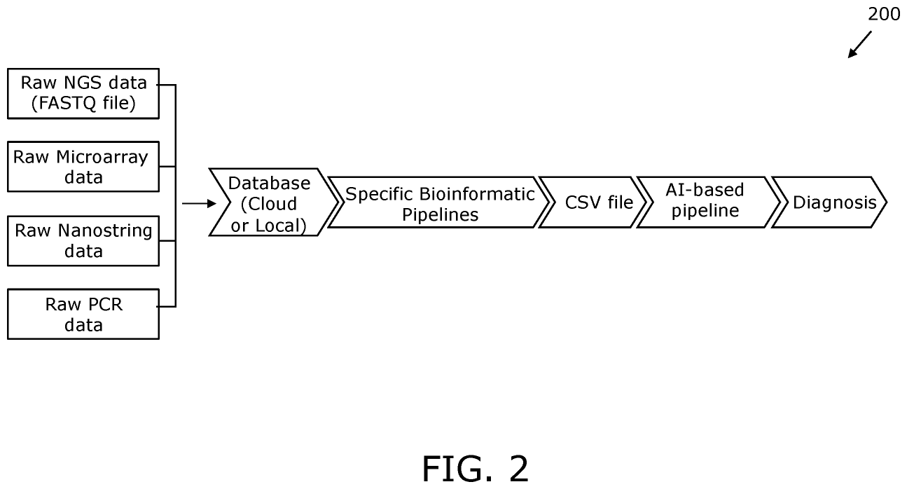

Referring to FIG. 2, shown is a schematic illustration 200 of steps of a method for multi-cancer management in a subject, in accordance with another embodiment of the invention. As shown, a miRNA expression profiling raw data is obtained from any of: Next Generation Sequencing technology, Polymerase Chain Reaction technology, Nanostring technology, Microarray technology. The obtained data may be stored in a database, wherein the database may be a cloud database or a local database. From the database, the miRNA expression profiling raw data is subjected to a standardized bioinformatic pipeline specific for any of the Next Generation Sequencing technology, Polymerase Chain Reaction technology, Nanostring technology, Microarray technology from which the miRNA expression profiling raw data is obtained. The standardized bioinformatic pipeline is configured to convert the miRNA expression profiling raw data into a formatted data (CSV file). The formatted data is received as an input in an artificial intelligence-based pipeline (proprietary “i-Biomarker”). The artificial intelligence-based pipeline is configured to use the formatted data for multi-cancer early detection and diagnosis in the subject.

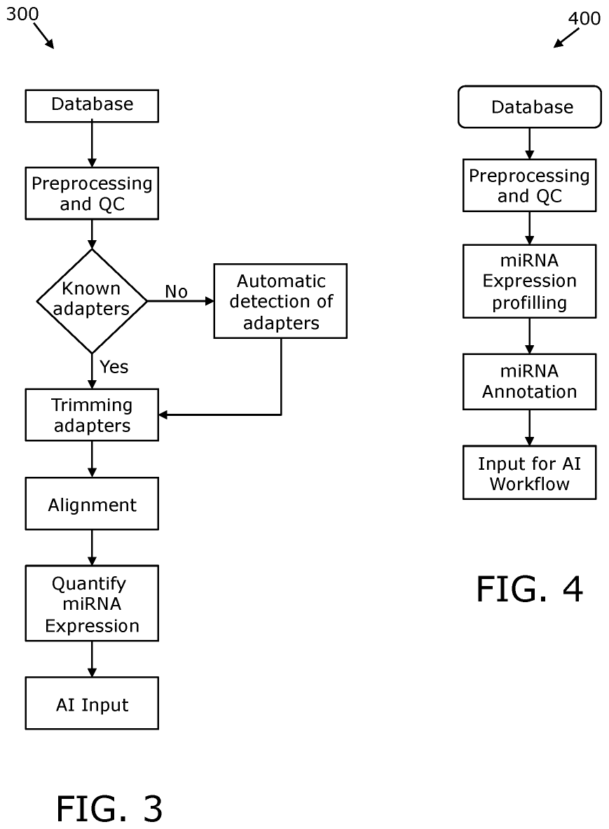

Referring to FIG. 3, shown is a flowchart 300 of a miRNA expression profiling analysis using the Next Generation Sequencing technology, in accordance with an embodiment of the invention. As shown, during the miRNA expression profiling analysis using the Next Generation Sequencing technology, the miRNA expression profiling raw data (FASTQ format) stored in the database is subjected to pre-processing and a quality control step. At this step, a report, with all information related to the sequences, is produced and analyzed, and decisions are taken regarding any further filtering steps for filtering out the molecules that are not miRNA. The filtered sequences are searched for known adaptors, or if the adapters are unknown specialized algorithms that can identify the adapters automatically are used. The identified adapters are then trimmed. The pre-processed, quality checked and trimmed sequences are aligned with a reference genome using the best performing alignment algorithms and then annotated using the most recent version of miRNA databases (e.g., miRBase). An algorithm will collapse all the identical sequences, count them, and then introduce them in a data frame during a quantification of the miRNA expression profiles. The data frame will then be completed with the number of sequences from each sample resulting in the complete expression profile for all miRNA for the entire experiment. The miRNA expression profile will then be used as in input in an artificial intelligence (AI) based pipeline (workflow). At this stage, an Exploratory Data Analysis or functional analysis step may be performed.

Referring to FIG. 4, shown is a flowchart 400 of a miRNA expression profiling analysis using the Microarray technology, in accordance with an embodiment of the invention. The process starts with raw data visualization to check the quality using various software packages. During the preprocessing and quality check step, the presence of technical variation between the array slides within the same experiment is checked and normalization of raw data is performed. At this step, the columns with null values (“NA”) will be identified and removed. During the miRNA expression profiling step, various open-source packages may be used. During the miRNA annotation step, the miRNA expression profile table requires the linking of the IDs of each molecule with the miRNA name specific to the platform used and the version of the miRNA database used by the equipment (e.g., miRBase). Therefore, this step oversees the correct annotation of each miRNA to every molecule ID from the expression table. This new matrix file becomes the input file that is introduced in the AI-based pipeline workflow.

Referring to FIG. 5, shown is a flowchart 500 of a miRNA expression profiling analysis using the Polymerase Chain Reaction technology, in accordance with an embodiment of the invention. During preprocessing Raw qPCR data step, the miRNA expression profiling raw data reported by the qPCR equipment and stored in the database is usually presented in the form of Ct values. Notably, the number of amplification rounds required for the fluorescence of a specific target gene to surpass an arbitrary threshold determines the Ct value. A gene expressed at a high level will have a low Ct value and one expressed at a low level will have a high Ct value, therefore the Ct value is inversely proportional to expression levels for the molecule of interest. There are various quality control (QC) filters that can be applied before the normalization process, each package having a specific method for finishing the QC step. During the normalization step, the qPCR data is normalized using various normalization (such as housekeeping genes-based normalization, rank-invariant set normalization and quantile normalization) algorithms. The new output file becomes the input file that is introduced in the AI-based pipeline workflow.

Referring to FIG. 6, shown is a flowchart 600 of a miRNA expression profiling analysis using the Nanostring technology, in accordance with an embodiment of the invention. Notably, for interpreting the miRNA expression profiling raw data produced by the Nanostring equipment-specific software (e.g., nSolver 4.0) must be used to open the text files containing the counts for each gene (e.g., RCC format, CSV format) on multiple operating systems. During Quality Check & Data Annotation step, the miRNA expression profiling raw data is imported from the database, and after importing the miRNA expression profiling raw data a selection of the QC parameters (such as Imaging QC, Binding Density QC, Positive Control Linearity QC and Positive Control Limit of Detection QC, depending on the analyte types detected in the data, ad other additional QC parameters) must be made. The data must be examined to ensure that counts of positive and negative controls along with housekeeping/endogenous genes meet expectations. Particularly for the samples marked with QC flags. In this step, the sample group names can also be assigned for the experiment. This step further allows to add the diagnosis column (with labels such as “Cancer” and “Control”) to the data. During the normalization step, a two-step data transformation, that balances counts between lanes, is used to normalize raw gene expression data. Herein, normalizing raw gene expression data includes a positive control normalization calculated with the positive controls spiked in every sample, and a CodeSet Content Normalization factor (HouseKeeping Normalization) calculated using reference genes to adjust for differences in analyte abundance or quality across all samples. Samples with unusually low count numbers for the previously mentioned methods can receive Normalization flags, and it's important to check if these counts meet the expectations. The software used provides an option to export the data in multiple formats. The preprocessed and normalized data will become the input for the AI-based pipeline workflow.

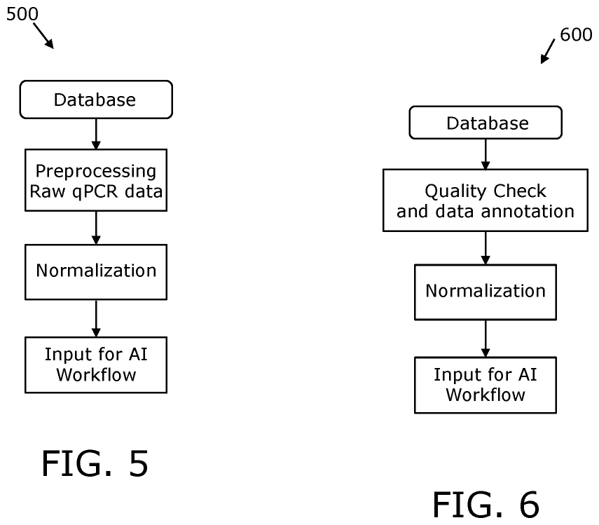

Referring to FIG. 7, shown is a flowchart 700 of a method of applying an AI-based pipeline workflow for developing and training new Models, in accordance with an embodiment of the invention. Herein, the raw data from the four technologies (Next Generation Sequencing technology, Polymerase Chain Reaction technology, Nanostring technology, Microarray technology) is processed by the corresponding bioinformatics pipelines, convert the data as above, and then apply the AI/ML pipeline. The output is a new model or an improved one.

The first step of the AI based pipeline starts with verifying the shape of the data observing the lines and columns. One of the columns will become the target for the prediction using AI algorithms. The input and output data must have the correct format, the miRNA columns will contain numeric variables and the target—the diagnosis—will be a categorical variable (namely, an output). Missing data and their percentage will also be identified in this step along with other possible anomalies and will be analyzed with a statistical summary (data distribution, minimum and maximum values, etc.). Later the correlation between the present variables will be checked, in this way the potential for some of the variables to be more important in the analysis and construction of the predictive models will be decided. Moreover, pre-processing data includes: identifying the missing values and inputting them with advanced methods; identifying Outliers through multiple methods and removing them; identifying if multiple names for the same column appear and correcting them, transforming the data by log 2 operations to bring the data closer to a Gaussian distribution; normalization through one of the following methods: scaling the data between values of 0 and 1, or between values of −1 and 1; standardization so that the average of the data becomes 0 and the standard deviation becomes 1.

Moreover, predictive models development comprises choosing any adequate AI/ML algorithm (e.g., Ensemble of Decision trees, Support Vector Machine, Shallow or Deep Neural Networks, etc.) or to use AutoML to develop more or less automatically the predictive models. Certain settings will still be required from the user, and could be introduced using, for example, a notebook environment (e.g., Jupyter Notebooks). The libraries along with the required packages necessary for the work environment of the AI workflow are loaded in this step. Next, the input-output pairs are defined—the miRNA expression values (input) and the target column (diagnosis; output) with values “Cancer” and “Non-Cancer”. The training and testing sets are then established. If the number of cases allows it, there will be a third set defined (the validation set). When the number of cases is small, just one set for training and testing is defined using cross-validation for training. For example, if the cases for training are split in 10 subsets, the learning will take place on 9 of them and the testing will be performed on the 10^{th} set, then the subsets are permuted.

At the stage of hyperparameter tuning, every modeling algorithm has a series of hyperparameters (e.g., depth of decisional trees for ensembles of decision trees). The default values of these algorithms often can reach a less than maximum performance, yet they allow for optimization. For this purpose, the validation subset is used, which when there are fewer cases must be a subset of the training set. If the performance obtained is superior, then the corresponding values of the hyperparameters are retained for the algorithm.

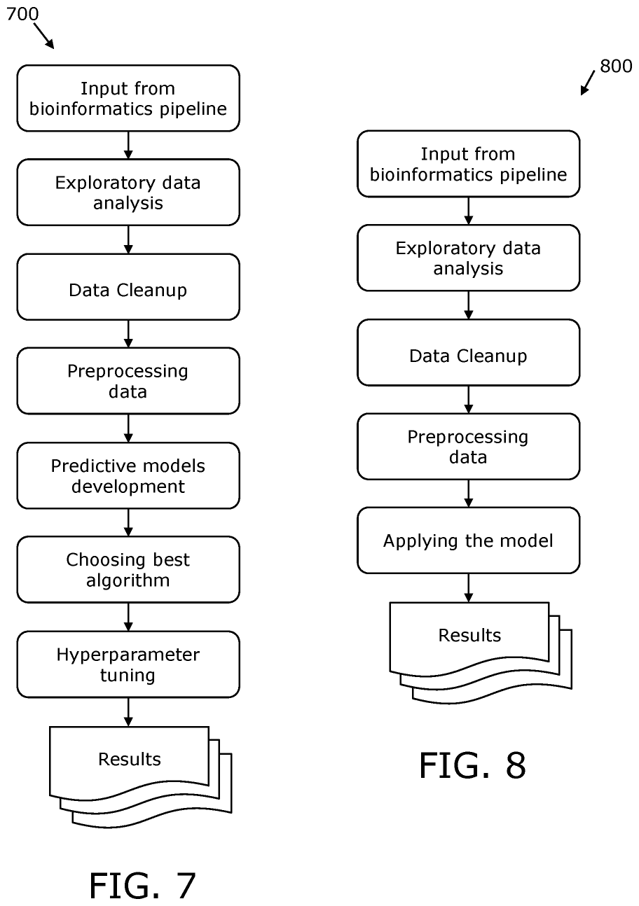

Referring to FIG. 8, shown is a flowchart 800 of a method of applying a trained AI-based pipeline workflow to the input data from the standardized bioinformatics pipeline, in accordance with an embodiment of the invention. As shown, the test (AI/ML model amalgamation, or combination) is applied to input data given by one of the four technologies (Next Generation Sequencing technology, Polymerase Chain Reaction technology, Nanostring technology, Microarray technology) to output a diagnosis and other information. Their main role is to transform the data into the format required by the AI/ML model amalgamation.

During Exploratory Data Analysis, the first step of the AI-based pipeline workflow starts with verifying the shape of the data observing the lines and columns. One of the columns will become the target for the prediction using the AI algorithms. The input and output data must have the correct format, the miRNA columns will contain numeric variables, and the target—the diagnosis-will be a categorical variable. Missing data and their percentage will also be identified in this step along with other possible anomalies and will be analyzed with a statistical summary (data distribution, minimum and maximum values, etc.). Later the correlation between the present variables will be checked, in this way the potential for some of the variables to be more important in the analysis and construction of the predictive models will be decided.

During preprocessing of the data step, the missing values are identified and inputted with advanced methods; outliers are identifying through multiple methods and removed; multiple names for the same column are identified and corrected; the data is transformed by log 2 operations to bring the data closer to a Gaussian distribution; the data is normalized through one of the following methods: scaling the data between values of 0 and 1, or between values of −1 and 1; the data is standardized so that the average of the data becomes 0 and the standard deviation becomes 1.

During Applying the predictive model step, the input pairs are defined—the miRNA expression values and the output—the target column (diagnosis) with values “Cancer” and “Non-Cancer”. The format is a table where each row represents a case, and each column represents a variable. One variable is the case ID, another is the target to be predicted (diagnostic here), and the rest are inputs. Certain settings will still be required from the user, and could be introduced using, for example, a notebook environment (e.g., Jupyter Notebooks). The libraries along with the required packages necessary for the work environment of the AI workflow are loaded in this step. Next, the input pairs are defined—the miRNA expression values and the output—the target column (diagnosis) with values “Cancer” and “Non-Cancer”.

1. A method for multi-cancer management in a subject, the method comprising:
(a) obtaining a miRNA expression profiling raw data;
(b) selecting a quality miRNA expression profiling raw data from amongst the obtained miRNA expression profiling raw data;
(c) converting the quality miRNA expression profiling raw data into a formatted data, wherein the formatted data comprises a table having a plurality of rows and a plurality of columns, wherein each row represents a case, and each column represents a variable, wherein the variable is
a case identification
an input represented by miRNA expression profiling raw data; and
an output representing a normal or a cancer of certain type pathologically confirmed diagnostic; and
(d) subjecting the formatted data to an artificial intelligence-based pipeline, for multi-cancer early detection and diagnosis in the subject, wherein the artificial intelligence-based pipeline is a trained artificial intelligence-based model or an explainable artificial intelligence-based model.

2. A method according to claim 1, further comprising repeating steps (a) to (d) at a pre-defined time interval for identifying an altered miRNA of the subject for monitoring a response to treatment.

3. A method according to claim 1, wherein monitoring the response to treatment results in a normal result or cancer recurrence, with a high confidence or a low confidence thereof.

4. (canceled)

5. A method according to claim 1, wherein the miRNA expression profiling raw data is obtained from any of: Next Generation Sequencing technology, Polymerase Chain Reaction technology, Nanostring technology, and Microarray technology.

6. A method according to claim 1, further comprising storing the miRNA expression profiling raw data in a database.

7. A method according to claim 1, further comprising subjecting the quality miRNA expression profiling raw data to a standardized bioinformatics pipeline specific for a miRNA expression profiling technology.

8. A method according to claim 1, further comprising performing at least one of: a quality control step, a filtering step, an adaptor trimming step, an alignment step, a quantification step, and a functional analysis step.

9. A method according to claim 1, wherein the formatted data is implemented as a CSV file.

10. (canceled)

11. (canceled)

12. A method according to claim 1, wherein the miRNA is circulating miRNA.

13. (canceled)

14. (canceled)

15. (canceled)

16. A system for multi-cancer management in a subject, the system comprising a processor configured to:
(a) obtain a miRNA expression profiling raw data;
(b) select a quality miRNA expression profiling raw data from amongst the obtained miRNA expression profiling raw data;
(c) convert the quality miRNA expression profiling raw data into a formatted data, wherein the formatted data comprises a table having a plurality of rows and a plurality of columns, wherein each row represents a case, and each column represents a variable, wherein the variable is
a case identification
an input represented by miRNA expression profiling raw data; and
an output representing a normal or a cancer of certain type pathologically confirmed diagnostic; and
(d) subject the formatted data to an artificial intelligence-based pipeline, for multi-cancer early detection and diagnosis in the subject, wherein the artificial intelligence-based pipeline is a trained artificial intelligence-based model or an explainable artificial intelligence-based model.

17. A system according to claim 16, wherein the processor is further configured to identify an altered miRNA of the subject for monitoring a response to treatment.

18. A system according to claim 16, wherein the processor is configured to result in a normal result or cancer recurrence, with a high confidence or a low confidence thereof, for monitoring the response to treatment.

19. (canceled)

20. A system according to claim 16, wherein the miRNA expression profiling data is obtained from any of: Next Generation Sequencing technology, Polymerase Chain Reaction technology, Nanostring technology, and Microarray technology.

21. A system according to claim 16, further comprising a database for storing the miRNA expression profiling raw data.

22. A system according to claim 16, wherein the processor is further configured to subject the quality miRNA expression profiling raw data to a standardized bioinformatics pipeline specific for a miRNA expression profiling technology.

23. A system according to claim 16, wherein the processor is further configured to perform at least one of: a quality control step, a filtering step, an adaptor trimming step, an alignment step, a quantification step, and a functional analysis step.

24. A system according to claim 16, wherein the formatted data is implemented in a CSV file.

25. (canceled)

26. (canceled)

27. (canceled)

28. (canceled)

29. A computer program product comprising a non-transitory computer readable storage medium having computer-readable instructions stored thereon, the computer-readable instructions being executable by a processor to execute a method as claimed in claim 1.
